(19) United States
(12) Patent Application Publication
(10) Pub. No.: US 2020/0276126 A1
(43) Pub. Date: Sep. 3, 2020

(54) POLYDOPAMINE-ENCAPSULATED NANODIAMONDS AND METHODS

(71) Applicant: THE UNITED STATES OF AMERICA, AS REPREESENTED BY THE SECRETARY, DEPARTMENT OF HEALTH AND HUMAN SERV, Bethesda, MD (US)

(72) Inventors: Haksung Jung, Rockville, MD (US); Keir Cajal Neuman, Bethesda, MD (US)

(21) Appl. No.: 16/650,507

(22) PCT Filed: Sep. 27, 2018
(86) PCT No.: PCT/US2018/053010

§ 371 Date: Mar. 25, 2020

Related U.S. Application Data
(60) Provisional application No. 62/565,552, filed on Sep. 29, 2017.

(51) Int. Cl. A61K 9/16; C09D 171/02; C01B 32/28; C09K 11/02; C09K 11/65; A61K 47/54; A61K 47/65

(52) U.S. Cl. CPC A61K 9/1676; C09D 171/02; C01B 32/28; C09K 11/025; C09K 11/65; B82Y 5/00; A61K 9/1611; A61K 47/545; A61K 47/65; C01P 2004/64; A61K 9/1641

(57) ABSTRACT

A method of preparing a polydopamine-coated nanodiamond includes contacting a nanodiamond with dopamine in an alkaline aqueous solution under conditions effective to form a polydopamine-coated nanodiamond. A method of functionalizing a nanodiamond includes preparing a polydopamine-coated nanodiamond; and covalently coupling a compound comprising a functional group to the polydopamine of the polydopamine-coated nanodiamond. A nanodiamond prepared by the methods is also disclosed.

### STATEMENT OF GOVERNMENT SUPPORT

This invention was made in part with government support from the National Institutes of Health. The government has certain rights in this invention.

### BACKGROUND

Nanoparticles have potential applications in a wide variety of fields, including biomedical, optical, and electronics. A nanoparticle is a particle having one or more dimensions of the order of 100 nanometer (nm) or less for which novel properties differentiate the nanoparticle from the bulk material.

Nanotechnology in medicine is making an impact in areas such as drug delivery systems, new therapies, in vivo imaging, nanoelectronics-based sensors, and neuroelectronic interfaces. Currently, there are relatively few (less than 10) types of core nanoparticles that are being modified and functionalized to be applied in these various applications.

Nanodiamonds are a type of nanoparticle having unique optical and magnetic properties. In particular, fluorescent nanodiamonds (FNDs) exhibit superb physical and chemical properties. FNDs show superior photostability with no photobleaching or blinking, and have near infrared (NIR) fluorescence (about 650 to 900 nm) with long fluorescence lifetimes (about 10-20 nanoseconds). Additionally, nanodiamonds have high biocompatibility and are chemically inert.

However, use of nanodiamonds has been limited thus far because of the difficulty in functionalizing or coating their inert surface. Their tendency to aggregate in aqueous solution further limits their use or functionalization for use.

Although several approaches to achieve surface modification of nanodiamonds have been reported, most of these methods require tedious surface treatment processes and can involve difficult chemical reactions.

Thus, there is a need for improved methods of modifying the surface of nanodiamonds.

### SUMMARY

A method of preparing a polydopamine-coated nanodiamond includes contacting a nanodiamond with dopamine in an alkaline aqueous solution under conditions effective to form a polydopamine-coated nanodiamond.

A method of functionalizing a nanodiamond includes preparing a polydopamine-coated nanodiamond; and covalently coupling a compound comprising a functional group to the polydopamine of the polydopamine-coated nanodiamond.

A nanodiamond prepared by the methods is also disclosed.

A polydopamine-coated nanodiamond comprises a nanodiamond core; and a polydopamine coating disposed at least partially on the nanodiamond core.

A surface-functionalized nanodiamond comprises a polydopamine-coated nanodiamond; and a compound covalently attached to a surface of the polydopamine coating of the polydopamine-coated nanodiamond, wherein the compound comprises a functional group.

These and other advantages, as well as additional inventive features, will be apparent from the following Drawings, Detailed Description, Examples, and Claims.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 presents a graph showing PDA shell thickness (in nanometers) around an 80 nm FND as a function of reaction time (minutes).

FIG. 2 is a photograph showing the increasing opacity of Table 3 reactions 1-5 correlating with increasing amounts of dopamine in the reaction and therefore increasing thickness of the PDA shell.

FIG. 3A and FIG. 3B are graphs showing the average size (diameter) and size distribution of PEGylated FND@PDA, determined by dynamic light scattering (DLS) with a Wyatt DynaPro NanoStar, as a function of weight of PEG modifying the surface of the PDA-encapsulated FND immediately after preparation (FIG. 3A) or after being stored in phosphate buffered saline (PBS) at room temperature for one week (FIG. 3B).

FIG. 4 is a graph of average particle size (diameter (nm)) of biotin-functionalized FND@PDA-PEG as a function of added streptavidin concentration (nM), illustrating the aggregation of FND@PDA-PEG-biotin at sub-saturating streptavidin concentrations (between vertical dotted lines), and dispersal of aggregates when streptavidin concentration is above the biotin concentration.

FIG. 5A is a graph of cell viability after a 16 hour incubation with increasing amounts of FND or FND@PDA-PEG in mouse immature BMDC or HeLa cells.

FIG. 5B is a graph of cell viability as a function of time of incubation with 100 μg/mL FND or FND@PDA-PEG in mouse immature BMDC or HeLa cells .

FIG. 6A presents three-channel confocal images of BMDCs incubated with uncoated FNDs. Each row corresponds to one cell imaged in three different channels and a merged image in the 4^{th} column. The white bar in the left-hand corner image represents 5 μm. The images in the first column are fluorescence from FNDs. The cells were co-stained with CD11c-FITC (column 2) and DAPI (column 3) to identify the membrane and nuclei, respectively, of the cells. FND (column 1) could not be internalized by BMDC cells due to the large size of aggregated FND resulting from poor colloidal stability in PBS buffer; see also column 4.

FIG. 6B presents three-channel confocal images of BMDCs incubated with FND@PDA-PEG. Each row corresponds to one cell imaged in three different channels and a merged image in the 4^{th} column. The white bar in the left-hand corner image represents 5 μm. The images in the first column are fluorescence from the FND@PDA-PEG. The cells were co-stained with CD11c-FITC (column 2) and DAPI (column 3) to identify the membrane and nuclei, respectively, of the cells. The FND@PDA-PEG (column 1) were internalized by BMDC cells; see also column 4, in which the FNDs can be observed as distinct spots inside the cells.

FIG. 7 is a TIRF microscopy image of streptavidin-conjugated-FND@PDA-PEG-biotin tethered to the biotinylated DNA on the sample cell obtained one month after preparation of the FND@PDA-PEG-biotin. The FND were excited at 532 nm and emission was collected with an Andor Xion 880 emCCD camera through a 560 nm long-pass filter.

FIG. 8A is a transmission electron microscopy (TEM) image of an uncoated 80 nm FND. The inset is a higher magnification EM image of an uncoated FND. Scale bar of inset image is 10 nm.

FIG. 8B is a graph of DLS measurements of uncoated 80 nm FND including the hydrodynamic diameter, the polydispersity percentage (PD %), which is a measure of the width of the size distribution, and the intensity of each peak in the population.

FIG. 8C is a graph showing the ultraviolet-visible (UV-Vis) absorption spectra of uncoated 80 nm FND and PDA-coated FND.

FIG. 8D is a graph of showing photoluminescence (PL) spectra of uncoated 80 nm FND and PDA-coated FND.

FIG. 9A and FIG. 9B present DLS results of FND@PDA and FND@PDA-PEG, respectively, in water. The polydispersity percentage (PD %) is a measure of the width of the size distribution.

### DETAILED DESCRIPTION

Disclosed herein is a method to encapsulate nanodiamonds in polydopamine. The polydopamine shell can readily be further functionalized by reaction with amine- or thiol-terminated molecules to generate surface-functionalized nanodiamonds. The inventors have found that the methods provide a simple, fast, and robust route for obtaining highly stable, monodisperse, and biocompatible surface-functionalized nanodiamonds. The stable, monodisperse surface-functionalized nanodiamonds can be used in various nanotechnology applications, including biomedical applications such as drug delivery, cell targeting, and imaging methods.

In an aspect, a method of preparing a polydopamine-coated nanodiamond is disclosed. The method comprises contacting a nanodiamond with dopamine in an alkaline aqueous solution under conditions effective to form a polydopamine-coated nanodiamond.

The aqueous solution can have a pH of 7.0 to 10.0, or 7.5 to 9.5, or 8.0 to 9.0, preferably 8.5.

The weight ratio of nanodiamond to dopamine can be 1:1 to 1:20, or 1:2.5 to 1:15, or 1:5 to 1:11.

The dopamine can be dopamine free base or dopamine hydrochloride.

In certain embodiments, the nanodiamond can be a fluorescent nanodiamond.

Contacting the nanodiamond with dopamine is performed at a temperature and for a time selected to obtain a polydopamine layer of a desired thickness. For example, the temperature can be 10 to 80° C., or 15 to 70° C., or 20 to 60° C., or 20 to 25° C. The contacting can be performed until the desired polydopamine shell thickness is obtained, for example for 10 minutes to 5 hours, or 15 minutes to 4 hours, or 20 minutes to 3 hours, or 30 minutes to 2 hours. The thickness of the polydopamine layer can be 2 to 20 nanometers (nm), or 2.5 to 16 nm, or 3 to 10 nm, as measured by transmission electron microscopy.

A “nanodiamond” refers to a nanodimensioned diamond particle. “Diamond” as used herein includes both natural and synthetic diamonds from a variety of synthetic processes, as well as “diamond-like carbon” (DLC) in particulate form. The diamond particles have at least one dimension of less than 1 micrometer, less than 800 nm, less than 500 nm, or less than 100 nm, for example 1 nm to about 100 nm or 1 to 500 nm. The particle can be of any shape, e.g., rectangular, spherical, cylindrical, cubic, or irregular, provided that at least one dimension is nanosized, i.e., less than 1 micrometer, less than 800 nm, less than 500 nm, or less than 100 nm. Nanodiamonds are commercially available. Alternatively, nanodiamonds can be prepared by methods known in the art. Nanodiamonds can be prepared, for example, by detonation of certain explosives in a closed container, laser ablation, high energy ball milling of diamond microcrystals, plasma-assisted chemical vapor deposition, or autoclave synthesis from supercritical fluids.

The term “fluorescent nanodiamond” (FND) refers to nanodiamonds that exhibit fluorescence when exposed to an appropriate absorption (excitation) spectrum. Fluorescent nanodiamonds are commercially available from a number of sources, e.g. Adámas Nanotechnologies (Raleigh, N.C.) or Sigma-Aldrich.

In another aspect, the polydopamine-coated nanodiamonds can be readily derivatized using methods known for derivatizing polydopamine. Such derivatization can be used to alter the physical characteristics of the polydopamine-coated nanodiamonds or to provide functionality for further derivatization or use. One method of covalently derivatizing a polydopamine surface is by covalently coupling a compound comprising a thiol, amine, or imidazole group to the surface via a Michael-type addition reaction or by Schiff base formation with an amine group of the compound. In certain embodiments, derivatization results in stabilization of the particle size and particle size distribution of the polydopamine-coated nanodiamonds in aqueous solution. Examples of suitable compounds that include or can be derivatized to include a thiol or amine group for coupling to the polydopamine-coated nanodiamond include polyethylene glycol (PEG), polyethylene glycol thiol, polyethylene glycol amine, dextran, poly(poly(ethylene glycol) methacrylate), serum albumin such as human serum albumin, DNA, folate, hyaluronic acid, D-a-tocopherol polyethylene glycol 1000 succinate, and poly(hydroxyethyl methacrylate), and derivatives thereof. In preferred embodiments, the compound is a PEG, or derivative thereof. The PEG can have a molecular weight of 1 kiloDalton (kDa) to 10 kDa, or 1.5 kDa to 7.5 kDa, or 2 kDa to 5 kDa.

Where the derivatization compound includes a functional group, the functional group can be further derivatized. Thus, it is also possible to use a functionalized derivatization compound as a linking group between the polydopamine surface and another molecule, such as a protein (e.g., streptavidin or an antibody) or other hydrophilic polymer (e.g., a nucleic acid, methyl cellulose, or poly(vinyl alcohol)). The functional group of the derivatization compound is selected to react with or bind specifically to the other molecule, and can be, for example, a vinyl, allyl, epoxy, acryloyl, methacryloyl, sulfhydryl, amino, hydroxy, biotin, or the like. The functionalization can be simultaneous or stepwise.

In a specific embodiment the polydopamine-coated nanodiamonds are chemically or physically functionalized to include a labeling material, a therapeutic agent, and/or targeting agent. The functionalization can be direct, or via a linker as described above.

The term “labeling material” refers to a material which is detectable by a physical or chemical method to permit identification of the location or quantity of the silica- coated nanodiamond. Detectable materials include fluorescent materials, dyes, light-emitting materials, radioactive materials, enzymes, and prosthetic groups. Examples of fluorescent materials include umbelliferone, fluorescein, fluorescein isothiocyanate, rhodamine, dichlorotriazinylamine fluorescein, dansyl chloride, or phycoerythrin. Examples of light-emitting materials include luminol, and examples of radioactive materials include ¹²⁵I, ¹³¹I, ³⁵S, and ³H. Examples of enzymes include horseradish peroxidase, alkaline phosphatase, β-galactosidase, or acetylcholinesterase. Examples of prosthetic groups include streptavidin/biotin and avidin/biotin. Detection of the labeling material can be performed by a method known in the art.

The therapeutic agent can be any known in the art. In an embodiment, the therapeutic agent is an anti-inflammatory agent, an antidiabetic agent, a chemotherapeutic agent, or an anti-angiogenesis agent.

The targeting agent can be a molecule that directs the nanodiamond to a specific cell type. For example, the targeting agent can be a ligand that specifically binds with a receptor found on the surface of a particular cell type of interest or a molecule that is selectively transferred within a particular cell type of interest.

The polydopamine-coated nanodiamonds can optionally be purified. Purifying the polydopamine-coated nanodiamonds from the dopamine or derivatizing compounds can be performed in a variety of ways. In an embodiment, unreacted reaction components are washed away from the polydopamine-coated nanodiamonds by successive rounds of centrifugation, removal of the supernatant, and resuspension in water or a desired solution, such as phosphate buffered saline (PBS). Sufficient rounds of centrifugation, supernatant removal, and resuspension are performed to obtain the desired degree of separation from unreacted reaction components.

In another aspect, a surface-functionalized nanodiamond is disclosed.

The surface-functionalized nanodiamond comprises a polydopamine-coated nanodiamond; and a compound covalently attached to a surface of the polydopamine coating of the polydopamine-coated nanodiamond, wherein the compound comprises a functional group. The functional group can be present on the surface of the surface-functionalized nanodiamond in an amount of at least 50 functional groups/nanodiamond, at least 100, at least 300, at least 500, at least 700, or at least 900, preferably 300 to 500 functional groups/nanodiamond. A surface density of 300 to 500 functional group/nanodiamond can also be expressed as one functional group per 40-70 nm² for a nanodiamond 80 nm in diameter. Examples of the functional group include a carboxylic acid, an acyl halide (e.g., an acyl chloride), an amide, a PEGylate, a biotinylate, or an amine. The functional group can be further derivatized to a second functional group. Examples of the second functional group include carboxylic acid or reactive derivative thereof, such as an anhydride, ester, or acyl halide (e.g., an acyl chloride), an amide, a PEGylate, a biotinylate, a folate, a thiol, a maleimide, an amine, a chelated gadolinium, an azide, an alkyne, a protein tag ligand, a double or single stranded DNA or RNA molecule, a peptide, or a dendrimer linkage. Examples of commercially available protein tag systems include HALOTAG (a modified haloalkane dehalogenase, Promega Corporation) and SNAP-TAG (a 20 kDa mutant of O⁶-alkylguanine-DNA alkyltransferase) and its derivative CLIP-TAG (both from New England Biolabs, Inc.). Examples of ligands for these protein tag systems include compounds comprising a haloalkane moiety for the HALOTAG system; compounds comprising an O⁶-alkylguanine moiety for the SNAP-TAG system, and compounds comprising an O²-benzylcytosine moiety for the CLIP-TAG system.

As is known in the art, accurate determination of particle dimensions in the nanometer range can be difficult. In an embodiment, the dimension of the nanodiamonds is determined using their hydrodynamic diameter. The hydrodynamic diameter of the nanodiamond or an aggregate of nanodiamonds can be measured in a suitable solvent system, such as an aqueous solution. The hydrodynamic diameter can be measured by sedimentation, dynamic light scattering, or other methods known in the art. In an embodiment, hydrodynamic diameter is determined by differential centrifugal sedimentation. Differential centrifugal sedimentation can be performed, for example, in a disc centrifuge. In an embodiment, the hydrodynamic diameter is a Z-average diameter determined by dynamic light scattering. The Z-average diameter is the mean intensity diameter derived from a cumulants analysis of the measured correlation curve, in which a single particle size is assumed and a single exponential fit is applied to the autocorrelation function. The Z-average diameter can be determined by dynamic light scattering with the sample dispersed in, for example, deionized water. An example of a suitable instrument for determining particle size and/or the polydispersity index by dynamic light scattering is a Malvern Zetasizer Nano.

The surface-functionalized polydopamine-coated nanodiamonds produced are monodisperse (e.g. show a relatively narrow monomodal lognormal particle size distribution with a polydispersity index of ≤0.4, ≤0.3, or ≤0.2) and stable in aqueous solution at room temperature for extended periods of time, for example at least 24 hours, at least 48 hours, at least 7 days, or at least one month. Such stability is improved when the pH of the aqueous solution is maintained at least at 2 and no more than 12, for example pH 2.0 to pH 12.0, pH 3.0 to pH 11.5, pH 4.0 to pH 11.0, pH 5.0 to pH 10.5, pH 6.0 to pH 10.0, pH 7.0 to pH 9.5, or pH 7.4 to pH 9.0.

Dispersity is a measure of the heterogeneity of sizes of molecules or particles in a given sample. “Monodisperse” refers to particles of the same or a similar size, while “polydisperse” refers to particles with a heterogeneous (e.g. multimodal) size distribution. The “polydispersity index” is a measure of the heterogeneity of the size distribution. For a size distribution determined by dynamic light scattering, the polydispersity index (PDI) is the width of the size distribution determined from the correlation function. Herein, an aqueous sample with a PDI≤0.4, specifically ≤0.35, more specifically ≤0.3, and yet more specifically ≤0.2 is considered to be monodisperse.

The following examples are merely illustrative of the methods disclosed herein, and are not intended to limit the scope hereof.

### EXAMPLES

### Methods and Chemicals

Dopamine hydrochloride (DA), tris(hydroxymethyl)aminomethane (TRIS), streptavidin, WST-1 Cell Proliferation Reagent (WST-1), ammonium hydroxide solution (NH₄OH, 25%), gentamicin, and 2-mercaptoethanol were purchased from Sigma-Aldrich. Reagents mPEG-SH (2 kDa) and biotin-PEG-SH (2 kDa,) were purchased from NANOCS. RPMI 1640 medium was purchased from Lonza. L-glutamine was purchased from Gibco. 10% heat-inactivated fetal calf serum was purchased from BioScience. Granulocyte/macrophage colony-stimulating factor (GM-CSF) was purchased from Peprotech. Anti-mouse CD11c-FITC was purchased from eBioscience. Poly-L-lysine was purchased from Invitrogen. Fluorescent nanodiamonds (FNDs) were supplied by Adamas Nanotechnologies. Deionized (DI) water with a resistivity of 18.2 MΩ·cm was from a Milli-Q Water Purification System.

### Example 1. Synthesis of Polydopamine-Coated Fluorescent Nanodiamonds

Dopamine hydrochloride was dissolved in 10 mM Tris-HCl (pH=8.5). FNDs (average particle size=80 nm) in suspension were added and the reaction proceeded at room temperature (25° C.) for a determined amount of time. The reaction mixture was then centrifuged for 15 min at 20,000 rpm, and the precipitate was re-dispersed in 10 mL of deionized (DI) water. The recovered polydopamine-coated FND (“FND@PDA”) solution was then filtered through a 0.2-μm syringe filter to remove aggregates. Solution compositions, reaction times, and average PDA shell thickness are shown in Table 1. Average thickness of the PDA shell was determined by direct measurement of the shell thickness from EM images of PDA coated FNDs (n=10).

TABLE 1
Reaction conditions control PDA shell thickness

| | TRIS | FND | Dopamine | Dopamine | Dopamine | Reaction time | Average shell thickness |
|---|---|---|---|---|---|---|---|
| Entry | (mL) | (mg) | (μmol) | (g) | (mg) | (hour) | (nm) |
| 1 | 9.53 | 0.2 | 5.9 | 0.000903762 | 0.90 | 2 | 2.7 ± 0.8 |
| 2 | 9.53 | 0.2 | 7.3 | 0.001118214 | 1.1 | 2 | 3.0 ± 1.0 |
| 3 | 9.53 | 0.2 | 9.2 | 0.001409256 | 1.4 | 2 | 6.9 ± 2.4 |
| 4 | 9.53 | 0.2 | 11.4 | 0.001746252 | 1.7 | 2 | 9.1 ± 1.6 |
| 5 | 9.53 | 0.2 | 14.3 | 0.002190474 | 2.2 | 0.5 | 2.9 ± 0.5 |
| 6 | 9.53 | 0.2 | 14.3 | 0.002190474 | 2.2 | 1 | 5.2 ± 1.2 |
| 7 | 9.53 | 0.2 | 14.3 | 0.002190474 | 2.2 | 1.5 | 11.3 ± 1.3 |
| 8 | 9.53 | 0.2 | 14.3 | 0.002190474 | 2.2 | 2 | 15.4 ± 3.5 |

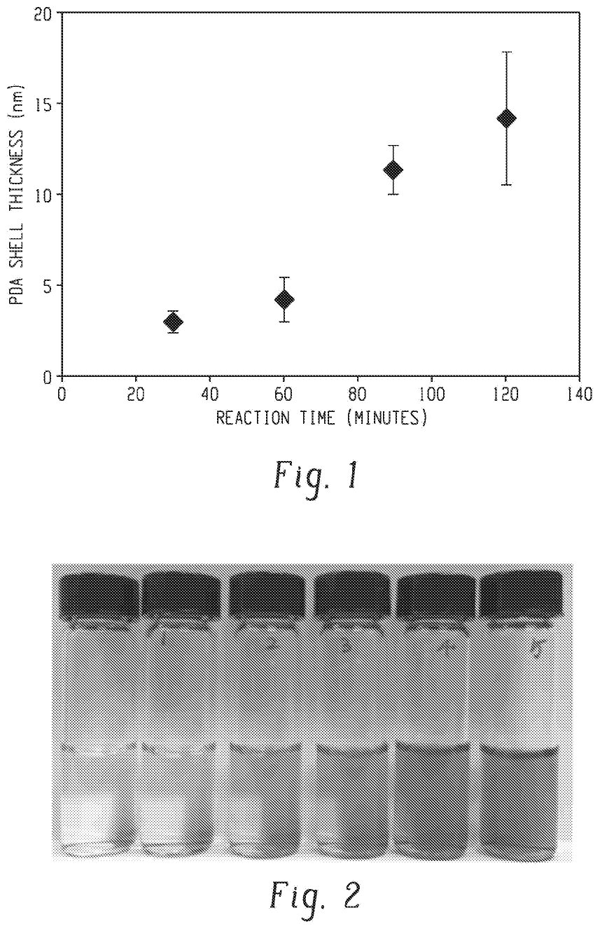

As can be seen in Table 1, thickness of the PDA shell around the 80 nm FNDs was observed to depend on the amount of dopamine and on the reaction time. The dependence of shell thickness on reaction time is graphed in FIG. 1. When the reaction proceeded for 30 minutes, the average shell thickness was 2.9±0.5 nm, while after 120 minutes of reaction time the average shell thickness was 15.4±3.5 nm. Thus, the PDA shell thickness can be controlled by varying the reaction time and/or the dopamine concentration.

Similar experiments were performed at room temperature to PDA encapsulate other commercial FNDs having an average particle size of 20, 40, 80, or 150 nm. Reaction conditions and PDA shell thickness are summarized in Table 2.

TABLE 2
Reaction conditions and PDA shell thickness
for FNDs of various diameter.

| Size of | 10 mM TRIS, | | Dopamine, | Reaction | PDA shell |
|---|---|---|---|---|---|
| FND | pH 8.5 | FND | 52.7 mM | time | thickness |
| (nm) | (μL) | (mg) | (μL) | (hour) | (nm) |
| 20 | 9530.2 | 0.2 | 269.8 | 1 | 4.0 ± 1.0 |
| 40 | 9530.2 | 0.2 | 269.8 | 1 | 4.4 ± 1.0 |
| 80 | 9530.2 | 0.2 | 269.8 | 1 | 4.2 ± 0.7 |
| 150 | 9530.2 | 0.2 | 269.8 | 1 | 7.5 ± 1.4 |

Under the same reaction conditions, the thickness of the PDA shell formed is relatively independent of the average FND diameter.

Solutions of PDA-coated FND were observed to get darker as a function of the PDA shell thickness. Five different FND encapsulation reactions with increasing amounts of dopamine, but otherwise identical were run, as shown in Table 3 below. The diameter of the FND was 80 nm.

TABLE 3
Reaction conditions for FIG. 2 reactions

| Sam- | Tris- | | | Reaction | Total |
|---|---|---|---|---|---|
| ple | HCl | FND | Dopamine | time | Volume |
| # | (μL) | (μL) | (μL) | (h) | (mL) |
| 1 | 9714.6 | 200 (0.2 mg) | 85.4 (4.5 μmol) | 2 | 10 |
| 2 | 9686.1 | 200 (0.2 mg) | 113.9 (6.0 μmol) | 2 | 10 |
| 3 | 9648.2 | 200 (0.2 mg) | 151.8 (8.0 μmol) | 2 | 10 |
| 4 | 9597.6 | 200 (0.2 mg) | 202.4 (10.7 μmol) | 2 | 10 |
| 5 | 9530.2 | 200 (0.2 mg) | 269.8 (14.3 μmol) | 2 | 10 |

FIG. 2 is a photograph showing the increasing opacity of reactions 1-5 correlating with increasing amounts of dopamine in the reaction and therefore increasing thickness of the PDA shell.

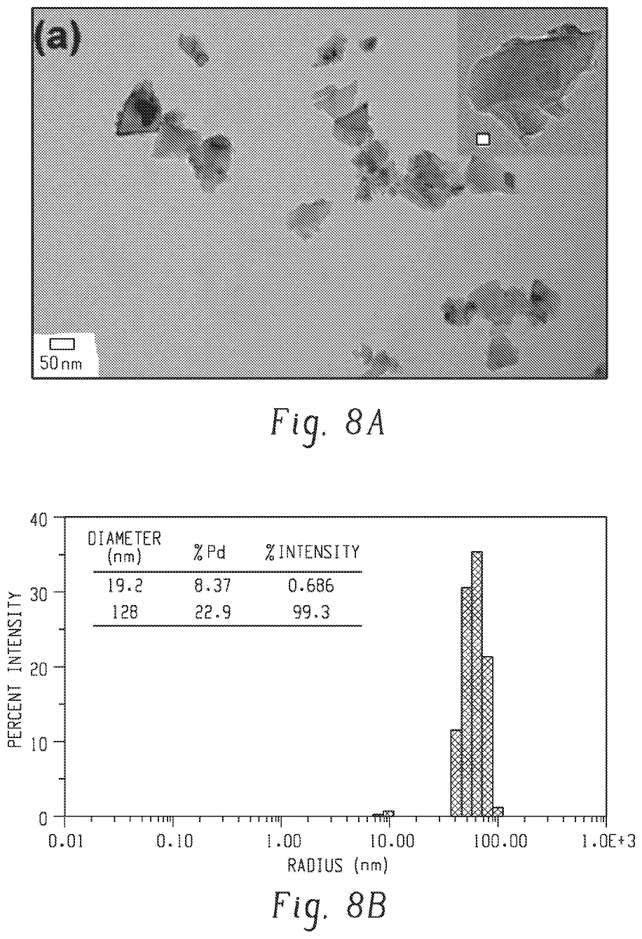

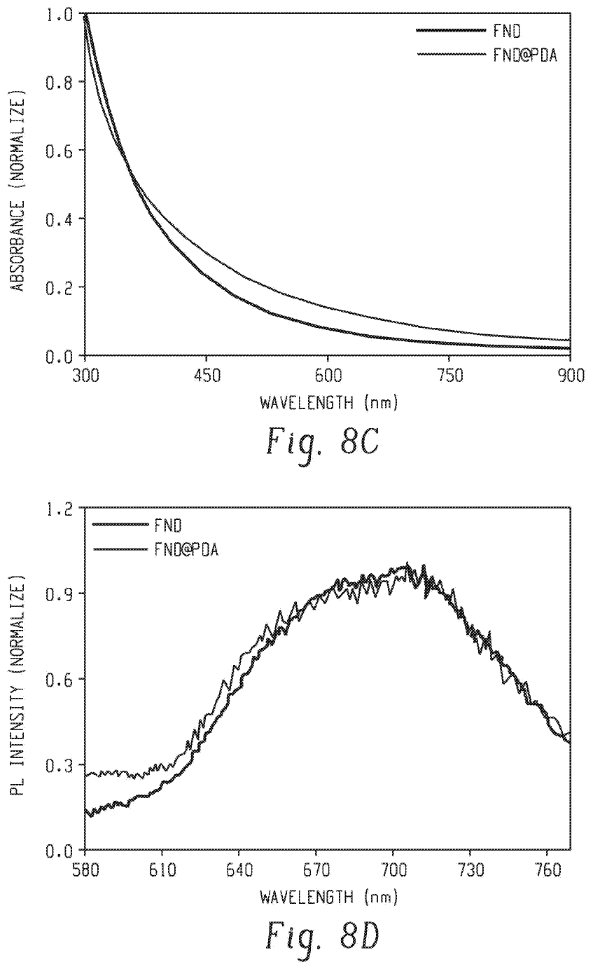

Uncoated FNDs exhibit irregular shapes with sharp edges, a broad size distribution, and their diameter measured by DLS is 128 nm (FIGS. 8A and 8B). Coating the FNDs with the PDA shell did not significantly alter the absorption or emission spectra of FNDs, as shown in FIGS. 8C and 8D, respectively.

### Example 2. PEGylation of PDA-Encapsulated FNDs

Surface modification of FND@PDA was performed by adding 2 kiloDalton (kDa) methoxy polyethylene glycol thiol (mPEG-SH) (4 mg) and 2 μL of NH₄OH (25%) into a solution of FND@PDA (0.2 mg of FND@PDA in 10 mL of DI water).

The mixture was stirred for 24 hours (h) at room temperature. The PEG-modified FND@PDA (“FND@PDA-PEG”) was isolated by centrifugation for 20 min at 20,000 rpm and the supernatant was removed. The isolated FND@PDA-PEG was then re-dispersed in water. This process of centrifugation of the FND@PDA-PEG dispersion followed by removal of the supernatant and re-dispersing the FND@PDA-PEG in water was repeated 2 times.

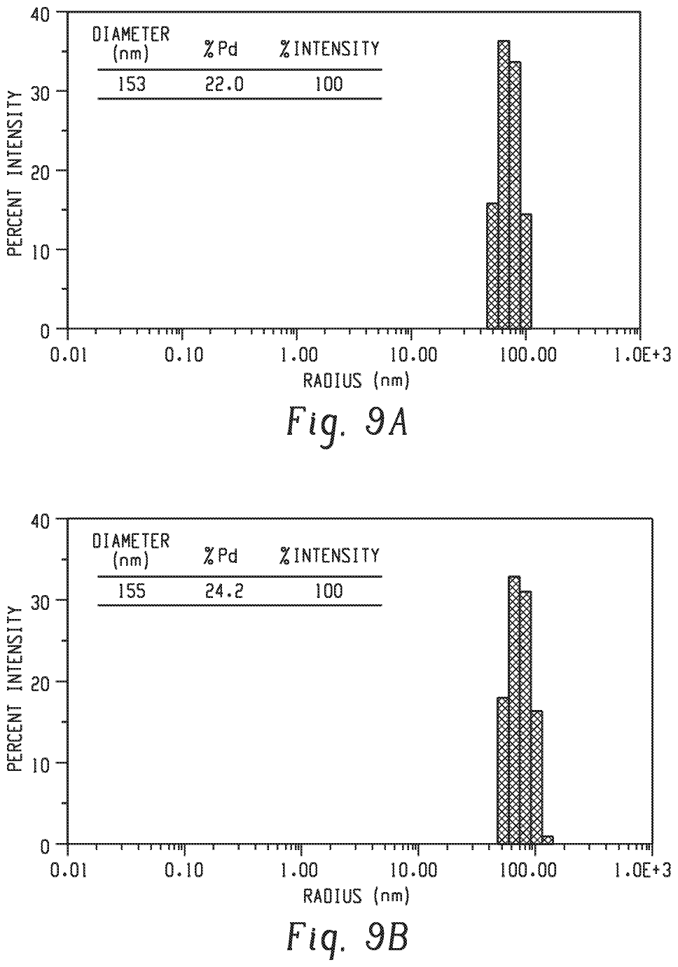

FIGS. 9A and 9B show that the diameter of the FND@PDA before and after PEGylation is comparable (153 nm vs 155 nm).

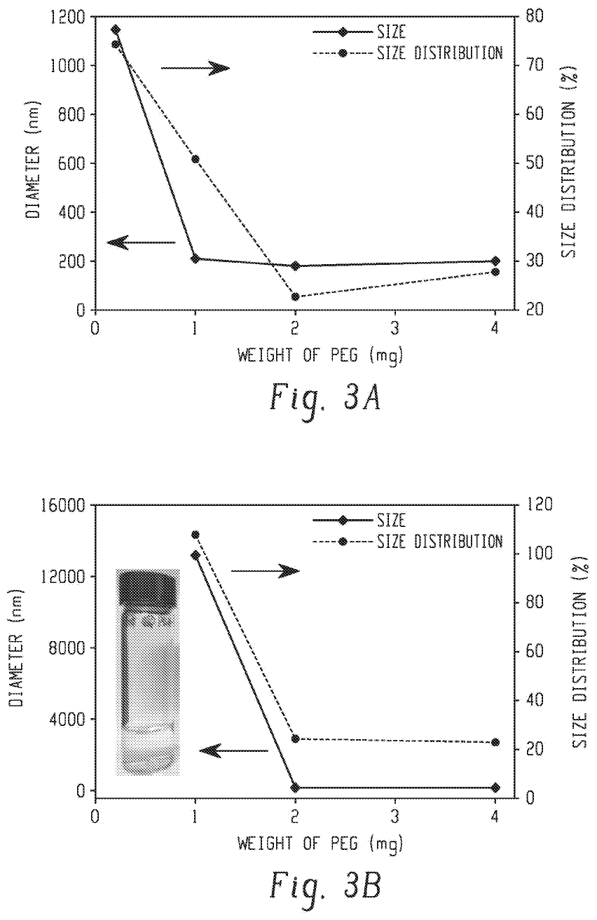

PEGylation of the FND@PDA was found to stabilize the size and size distribution of the FND@PDA particles in phosphate buffered saline (PBS). FIG. 3A shows the particle size and size distribution measured for FND@PDA immediately after being PEGylated with varying amounts of PEG, washed, and then dispersed in PBS. With use of at least 2 mg PEG per 0.2 mg FND@PDA, the average particle size and size distribution of the FND@PDA-PEG particles was independent of the amount of PEG. Below 2 mg PEG per 0.2 mg FND@PDA, agglomeration of particles occurs and polydispersity is greater. FIG. 3B shows the particle size and size distribution measured for the same preparation of FND@PDA-PEG after storage in PBS for one week a room temperature. With use of at least 2 mg PEG per 0.2 mg FND@PDA, the average particle size and size distribution of the FND@PDA-PEG particles was independent of the amount of PEG, while below 2 mg PEG per 0.2 mg FND@PDA, agglomeration of particles is observed and polydispersity is greater.

As demonstrated below, the FND@PDA-PEG particles have been shown to be stable after storage in PBS at room temperature for at least four months.

### Example 3. Biotinylation of PDA-Encapsulated FNDs

PDA-encapsulated FNDs were modified using a biotin polyethylene glycol thiol (Biotin-PEG-SH). The structure of biotin-PEG-SH is shown below.

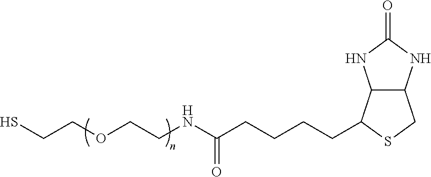

The biotin-PEG-SH used in these experiments had a molecular weight of 2 kiloDaltons (kDa) and n=39.

2 μL of NH₄OH solution (25%) was added to 10 mL of dispersed FND@PDA solution (0.02 mg/mL in DI water) to adjust the pH of the solution to ˜10.3. To this mixture, 4 mg of biotin-PEG-SH was added. After vigorous agitation for 24 h, biotin-PEG-modified FND@PDA (“FND@PDA-PEG-biotin”) was retrieved by centrifugation (20,000 rpm) and washed with DI water two times by the redispersion/centrifugation methodology discussed above. The FND@PDA-PEG-biotin solution was filtered through a 0.2-μm syringe filter to remove aggregates.

The level of biotinylation of the FND @ PDA was determined by a streptavidin titration assay, previously described in U.S. Application No. 62/402,339 and WO2018/064504, which relies on the aggregation of biotinylated nanoparticles with the addition of sub-saturating amounts of streptavidin. Because the streptavidin is tetravalent for binding biotin, under conditions where all of the surface-bound biotins are not saturated with streptavidin, the nanodiamonds will aggregate. If the diamonds are mixed with increasing concentrations of streptavidin and the size of the particles is measured by dynamic light scattering after a suitable incubation period, then a characteristic curve is seen in which the size increases with increasing streptavidin concentration up to a critical concentration at which the measured size decreases to a value close to, but slightly larger than the original size. This critical concentration is the concentration of accessible biotin on the sample.

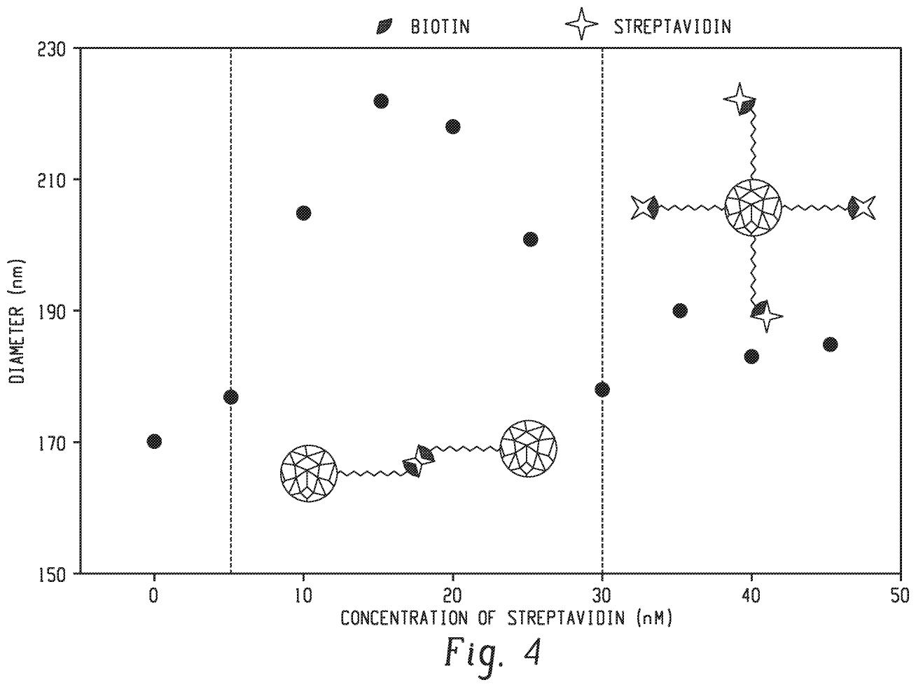

Cuvette-based dynamic light scattering (DLS) with a Wyatt DynaPro NanoStar is used to determine the hydrodynamic radius of a 50 μl sample of 0.05 mg/ml modified diamonds suspended in phosphate buffered saline (PBS) buffer. Average particle size (intensity-weighted average hydrodynamic radius) is measured by regularization methods as a function of increasing concentration of streptavidin. Results for one experiment quantifying the number of biotins modifying the FND@PDA-PEG-biotin surface is shown in FIG. 4. Without streptavidin (0 nM in FIG. 4), the modified diamonds are dispersed and have a particular average particle size. For concentrations of streptavidin below the surface-bound concentration of biotin, the diamonds aggregate via streptavidin crosslinking, and the average particle size increases (region between the two vertical dotted lines). Once the streptavidin concentration is equal to the biotin concentration, the diamonds begin to disperse again and the average particle size decreases. The concentration of biotin is well approximated by the concentration of streptavidin at which the size decreases, indicated for the FND@PDA-PEG-biotin by the dotted line at 30 nM streptavidin in FIG. 4. The FND@PDA-PEG-biotin were found to have about 340 biotins per FND.

In control experiments with FND@PDA without biotinylation, the hydrodynamic radius of the particles remained constant over the same range of streptavidin concentration, indicating that aggregation in FIG. 4 is specific and indicative of surface-bound biotins.

### Example 4. Determination of Cell Toxicity and Ability to be Taken Up by Cells

Experiments were performed to assess the toxicity of FND and PEGylated FND@PDA on immature mouse bone marrow dendritic cells (BMDCs).

4×10⁶ bone marrow cells per well were cultured in tissue-culture-treated 6-well plates in 4 mL of complete medium (e.g. RPMI 1640 supplemented with glutamine), 10% heat-inactivated fetal calf serum, and granulocyte/macrophage colony-stimulating factor (GM-CSF) (20 ng/mL) using standard protocols (e.g., Inaba, K., et al. Generation of large numbers of dendritic cells from mouse bone marrow cultures supplemented with granulocyte/macrophage colony-stimulating factor. J. Exp. Med. 1992, 176, 1693-702). The culture medium was replaced with fresh warmed medium with GM-CSF (20 ng/mL) every 2 days. On day 7, non-adherent cells in the culture supernatant and loosely adherent cells harvested by gentle washing with PBS were pooled and used as the starting source of material for most experiments.

BMDC were harvested on day 7, and then cells were transferred to a 96-well tissue culture plate in a final volume of 100 μL/well culture medium. Cells were treated with various amounts of FND@PDA-PEG or FND in triplicate and incubated for a fixed amount of time. Cells were then treated with 10 μL cell proliferation reagent WST-1 (2-(4-iodophenyl)-3-(4-nitrophenyl)-5-(2,4-disulfophenyl)-2H-tetrazolium, monosodium salt), mixed gently, and incubated for 4 hours. Cell viability was determined from absorbance at 450 nm, measured as described in the manual provided by the kit manufacturer.

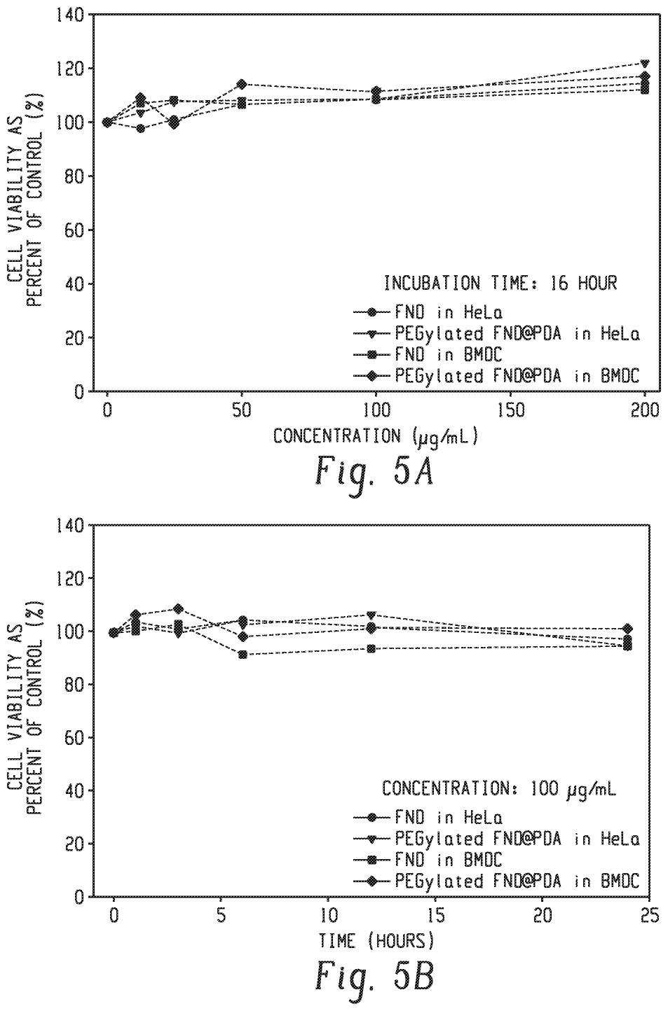

After a 16 hour incubation with increasing amounts of FND or FND@PDA-PEG, cell viability, determined as a percent of viability of control cells, was determined. The cell viability results as a function of FND or FND@PDA-PEG concentration are shown in FIG. 5A. At all conditions, no cell toxicity was observed.

Additional cell viability experiments were performed varying the exposure time of the cells to a fixed concentration (100 μg/mL) of FND or FND@PDA-PEG. The results of these experiments are shown in FIG. 5B. At all times, little loss of cell viability was observed.

As shown in FIGS. 5A and 5B, similar cell viability results were obtained in experiments using human cervical adenocarcinoma (HeLa) cells.

Confocal imaging was performed to determine whether the FND or FND@PDA-PEG could be taken up by the BMDCs.

BMDC were incubated with 50m/mL FND@PDA-PEG or FND for 16 hours at 37° C. and washed with PBS. Cells were stained with anti-mouse CD11c-FITC on ice for 30 min, washed, and then fixed with 4% paraformaldehyde in phosphate buffered saline (PBS) for 30 min at room temperature, and then washed twice with PBS. Fixed BMDCs were plated on poly-L-lysine-coated glass coverslips for 30 min at room temperature for analysis by confocal microscopy. Prior to imaging, ProLong Gold antifade reagent with DAPI was added to the slide. Cells were imaged using a Zeiss LSM 510 META confocal microscope using a ×63 oil immersion objective lens.

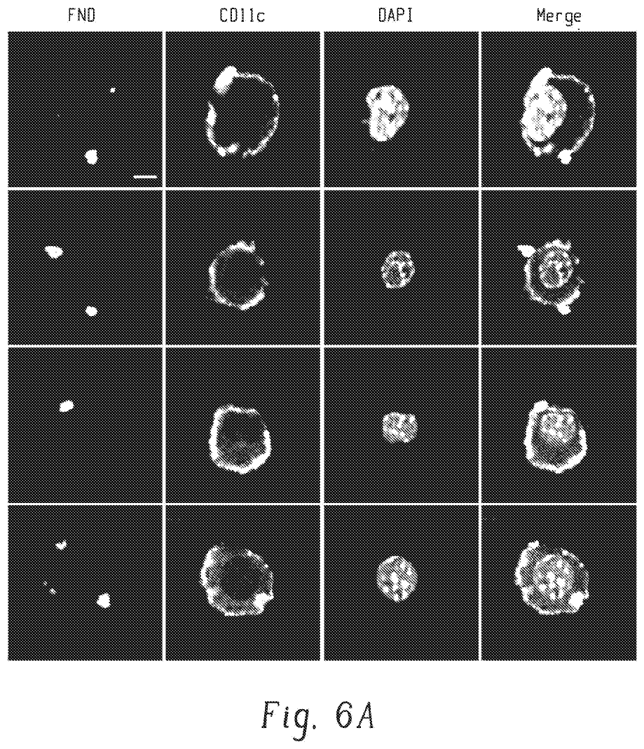

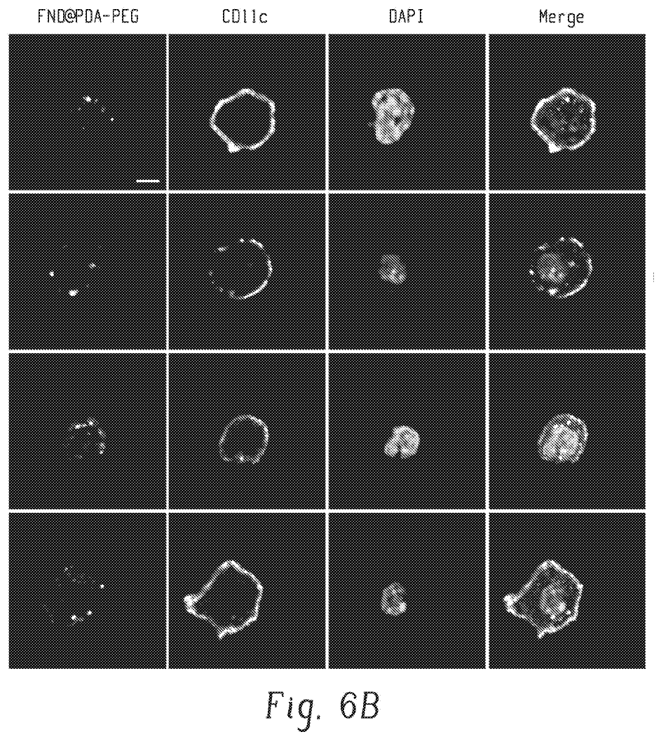

FIG. 6A presents three-channel confocal images of BMDCs incubated with uncoated FNDs. Each row corresponds to one cell imaged in three different channels and a merged image in the 4th column. The cells were co-stained with CD11c-FITC (column 2) and DAPI (column 3) to identify the membrane and nuclei, respectively, of the cells. FND (column 1) could not be internalized by BMDC cells due to poor colloidal stability in PBS buffer, see also column 4. FIG. 6B presents the same confocal data collected with BMDC incubated with FND@PDA-PEG. Individual PEG-FND@PDA were internalized by BMDC cells. See column 1 and also column 4, in which the PEG-FND@PDA can be observed as distinct spots within the cells.

Individual PEG-FND@PDA were also internalized by HeLa cells, while unmodified FNDs adhered to the cell membrane.

### Example 5. Preparation of Streptavidin Conjugated FND

Streptavidin-conjugated biotinylated FND@PDA was prepared by a simple binding reaction between biotinylated FND@PDA and streptavidin. 100 μL (90 nM) of streptavidin in Wash Buffer (WB; 1×PBS, 0.03% w/v BSA, and 0.01% v/v Tween-20) was added to a suspension of biotinylated FND@PDA (0.1 mg/mL) in water (100 μL). The mixture was agitated overnight. The resulting dispersion was centrifuged for 10 min at 12,000 rpm, and the precipitate was redispersed in 100 μL of WB.

### Example 6. DNA Tethering to Streptavidin-Conjugated Biotinylated FND@PDA

Streptavidin-conjugated biotinylated FND@PDA can be tethered to biotinylated DNA.

A detailed DNA tethering procedure and instrumentation for the single molecule fluorescence assay were previously published previously. (Seol, Y.; Neuman, K. C., Magnetic Tweezers for Single-Molecule Manipulation. In Single Molecule Analysis: Methods and Protocols, Peterman, E. J. G.; Wuite, G. J. L., Eds. Humana Press: Totowa, N.J., 2011; pp 265-293; Seol, Y., et al. A kinetic clutch governs religation by type IB topoisomerases and determines camptothecin sensitivity. Proc. Natl. Acad. Sci. USA 2012, 109, 16125-16130.)

In brief, 2.5 kb DNA labelled with biotin and digoxigenin was generated by PCR of pET28b DNA plasmid with 5′ biotin- and 5′ digoxin-primers respectively (Operon). 2.5 kb DNA molecules (25 pM) were incubated with anti-digoxigenin (25 nM) in 1×PBS for 15 min to allow anti-digoxigenin-digoxigenin binding. The DNA-anti-digoxigenin mixture was then added into a pre-assembled sample cell and incubated at 4° C. overnight to allow anti-digoxigenin to non-specifically absorb to the sample cell surface. The sample cell was then washed with 200 μl of WB to remove unbound DNA molecules.

FND-DNA tethers were formed by introducing 40 μl of streptavidin-conjugated-FND@PDA-PEG-biotin (0.1 mg/mL) into the sample cell followed by incubation overnight at 4° C. or 1 hour at RT. After washing with 600 μl of WB, FND-attached DNA tethers were visualized using a total internal reflection fluorescence (TIRF) microscope (excitation: 560 nm and emission: 640 nm).

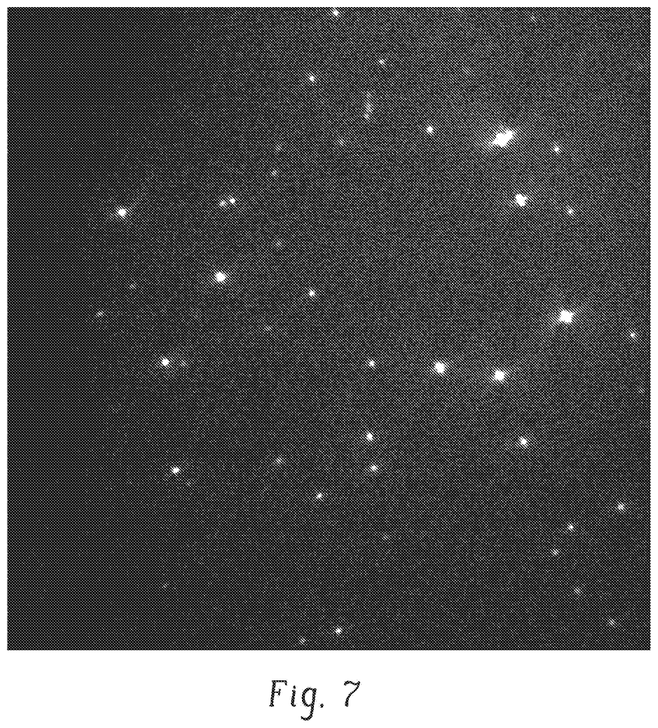

FIG. 7 is a TIRF microscopy image of streptavidin-conjugated-FND@PDA-PEG-biotin tethered to biotinylated DNA on the sample cell. The tethering experiment was done a month after preparation of the FND@PDA-PEG-biotin followed by storage in water at room temperature. A similar efficiency of DNA tethering was determined by TIRF imaging for the same batch of FND@PDA-PEG-biotin after four months storage in water at room temperature. This demonstrates the FND@PDA-PEG particles are stable after storage in water at room temperature for at least four months.

In summary, methods to generate polydopamine-coated nanodiamonds and surface-functionalized nanodiamonds made by the methods have been shown. The method is simple, robust, and reproducible and is able to generate highly stable and monodisperse surface-modified polydopamine-coated nanodiamonds. The surface-functionalized nanodiamonds are shown to have high levels of functional groups per nanodiamond and to have long-term chemical and physical stability in aqueous solution.

The invention is further illustrated by the following Aspects, which are not intended to be limiting.

Aspect 1. A method of preparing a polydopamine-coated nanodiamond, the method comprising contacting a nanodiamond with dopamine in an alkaline aqueous solution under conditions effective to form a polydopamine-coated nanodiamond.

Aspect 2. The method of aspect 1, wherein the contacting is at a temperature of 10 to 80° C., or 15 to 70° C., or 20 to 60° C., or 20 to 25° C.

Aspect 3. The method of aspect 1 or 2, wherein the contacting is for 10 minutes to 5 hours, or 15 minutes to 4 hours, or 20 minutes to 3 hours, or 30 minutes to 2 hours.

Aspect 4. The method of any one of aspects 1 to 3, wherein the solution has pH 2.0 to pH 12.0, pH 3.0 to pH 11.5, pH 4.0 to pH 11.0, pH 5.0 to pH 10.5, pH 6.0 to pH 10.0, pH 7.0 to pH 9.5, or pH 7.4 to pH 9.0.

Aspect 5. The method of any one of aspects 1 to 4, wherein the weight ratio of nanodiamond to dopamine is 1:1 to 1:20, or 1:2.5 to 1:15, or 1:5 to 1:11.

Aspect 6. The method of any one of aspects 1 to 5, wherein the nanodiamond is a fluorescent nanodiamond.

Aspect 7. The method of any one of aspects 1 to 6, further comprising derivatizing the polydopamine layer of the polydopamine-coated nanodiamond.

Aspect 8. The method of any of aspects 1 to 7, wherein derivatizing the polydopamine comprises covalently coupling a compound comprising a functional group to the polydopamine of the polydopamine-coated nanodiamond.

Aspect 9. The method of any of aspects 1 to 8, further comprising purifying the polydopamine-coated nanodiamond.

Aspect 10. A method of functionalizing a nanodiamond, comprising preparing a polydopamine-coated nanodiamond; and covalently coupling a compound comprising a functional group to the polydopamine of the polydopamine-coated nanodiamond.

Aspect 11. The method of any one of aspects 8 to 10, wherein covalent coupling is performed by a Michael-type addition reaction with a thiol or amine group of the compound or by Schiff base formation with an amine group of the compound.

Aspect 12. The method of any one of aspects 8 to 11, wherein the compound is polyethylene glycol , polyethylene glycol thiol, polyethylene glycol amine, dextran, poly(poly(ethylene glycol) methacrylate), serum albumin such as human serum albumin, DNA, folate, hyaluronic acid, D-α-tocopheryl polyethylene glycol 1000 succinate, and poly(hydroxyethyl methacrylate), a derivative of any of the foregoing, or a combination thereof.

Aspect 13. The method of any one of aspects 8 to 12, wherein the functional group is a carboxylic acid, a carboxylic acid ester, an anhydride, an acyl halide, an amide, a PEGylate, a biotinylate, a folate, a thiol, a maleimide, an amine, a chelated gadolinium, an azide, an alkyne, a protein tag ligand, a double or single stranded DNA or RNA molecule, a peptide, or a dendrimer linkage.

Aspect 14. A nanodiamond prepared by the method of any one of aspects 1 to 13.

Aspect 15. A polydopamine-coated nanodiamond comprising a nanodiamond core; and a polydopamine coating disposed at least partially on the nanodiamond core.

Aspect 16. A surface-functionalized nanodiamond comprising a polydopamine-coated nanodiamond; and a compound covalently attached to a surface of the polydopamine coating of the polydopamine-coated nanodiamond, wherein the compound comprises a functional group.

Aspect 17. The nanodiamond of aspect 15 or 16 which is a fluorescent nanodiamond.

Aspect 18. The nanodiamond of any one of aspects 15 to 17, wherein the functional group is a carboxylic acid, an acyl chloride, an amide, a pegylate, a biotinylate, a folate, a thiol, a maleimide, an active ester, an amine, a chelated gadolinium, an azide, an alkyne, a protein tag ligand, a double or single stranded DNA or RNA molecule, a peptide, or a dendrimer linkage.

Aspect 19. The nanodiamond of any one of aspects 15 to 18, wherein the functional group is further derivatized.

The compositions and methods can alternatively comprise, consist of, or consist essentially of, any appropriate components or steps herein disclosed. The compositions and methods can additionally, or alternatively, be provided so as to be devoid, or substantially free, of any steps, components, materials, ingredients, adjuvants, or species that are otherwise not necessary to the achievement of the function and/or objectives of the compositions and methods.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements can be combined in any suitable manner in the various embodiments.

Unless defined otherwise, technical and scientific terms used herein have the same meaning as is commonly understood by one of skill in the art to which this invention belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen can arise to applicants or others skilled in the art. Accordingly, the appended claims as filed and as they may be amended are intended to embrace all such alternatives, modifications variations, improvements, and substantial equivalents.

1. A method of preparing a polydopamine-coated nanodiamond, the method comprising
contacting a nanodiamond with dopamine in an alkaline aqueous solution under conditions effective to form a polydopamine-coated nanodiamond.

2. The method of claim 1, wherein the contacting is at a temperature of 10 to 80° C.

3. The method of claim 1, wherein the contacting is for 10 minutes to 5 hours.

4. The method of claim 1, wherein the solution has pH 7.0 to pH 10.0.

5. The method of claim 1, wherein the weight ratio of nanodiamond to dopamine is 1:1 to 1:20.

6. The method of claim 1, wherein the nanodiamond is a fluorescent nanodiamond.

7. The method of claim 1, further comprising
derivatizing the polydopamine layer of the polydopamine-coated nanodiamond.

8. The method of claim 7, wherein derivatizing the polydopamine comprises
covalently coupling a compound comprising a functional group to the polydopamine of the polydopamine-coated nanodiamond.

9. The method of claim 1, further comprising
purifying the polydopamine-coated nanodiamond.

10. A method of functionalizing a nanodiamond, comprising
preparing a polydopamine-coated nanodiamond; and
covalently coupling a compound comprising a functional group to the polydopamine of the polydopamine-coated nanodiamond.

11. The method of claim 10, wherein covalent coupling is performed by a Michael-type addition reaction with a thiol or amine group of the compound or by Schiff base formation with an amine group of the compound.

12. The method of claim 10, wherein the compound is polyethylene glycol, polyethylene glycol thiol, polyethylene glycol amine, dextran, poly(poly(ethylene glycol) methacrylate), a serum albumin, DNA, folate, hyaluronic acid, D-α-tocopheryl polyethylene glycol 1000 succinate, poly(hydroxyethyl methacrylate), a derivative of any of the foregoing, or a combination thereof.

13. The method of claim 12, wherein the functional group is a carboxylic acid, a carboxylic acid ester, an anhydride, an acyl halide, an amide, a PEGylate, a biotinylate, a folate, a thiol, a maleimide, an amine, a chelated gadolinium, an azide, an alkyne, a protein tag ligand, a double or single stranded DNA or RNA molecule, a peptide, or a dendrimer linkage.

14. (canceled)

15. A polydopamine-coated nanodiamond comprising
a nanodiamond core; and
a polydopamine coating disposed at least partially on the nanodiamond core.

16. A surface-functionalized nanodiamond comprising
the polydopamine-coated nanodiamond of claim 15; and
a compound covalently attached to a surface of the polydopamine coating of the polydopamine-coated nanodiamond, wherein the compound comprises a functional group.

17. The nanodiamond of claim 15 which is a fluorescent nanodiamond.

18. The nanodiamond of claim 16, wherein the functional group is a carboxylic acid, an acyl chloride, an amide, a pegylate, a biotinylate, a folate, a thiol, a maleimide, an active ester, an amine, a chelated gadolinium, an azide, an alkyne, a protein tag, a double or single stranded DNA or RNA molecule, a peptide, or a dendrimer linkage.

19. The nanodiamond of claim 16, wherein the functional group is further derivatized.
